(19) United States
(12) Patent Application Publication
(10) Pub. No.: US 2025/0052736 A1
(43) Pub. Date: Feb. 13, 2025

(54) METHODS FOR DETERMINING A SOURCE OF WOOD PRODUCTS, WOOD PRESERVATIVE COMPOSITIONS, WOOD PRODUCTS, AND METHODS OF TREATING WOOD SUBSTRATES

(71) Applicant: Koppers Performance Chemicals, Inc., Pittsburgh, PA (US)

(72) Inventors: Javier Romero, Peachtree City, GA (US); Douglas Herdman, Peachtree City, GA (US); Jun Zhang, Peachtree City, GA (US); Barry Sewell, Covington, GA (US)

(21) Appl. No.: 18/774,695

(22) Filed: Jul. 16, 2024

Related U.S. Application Data
(60) Provisional application No. 63/532,195, filed on Aug. 11, 2023.

(51) Int. Cl. G01N 33/46; B27K 3/22; B27K 3/26; G01N 21/78

(52) U.S. Cl. CPC G01N 33/46; B27K 3/22; B27K 3/26; G01N 21/78

(57) ABSTRACT

Methods for determining a source of wood products, wood products, wood preservative compositions, and methods of treating wood substrates are provided. The method comprises detecting a presence of a marker in the wood product and determining the source of the wood product based on the detected presence of the marker.

### CROSS-REFERENCE

This application claims priority to U.S. Provisional Patent Application No. 63/532,195 which was filed on Aug. 11, 2023. The contents of which is hereby incorporated by reference into this specification.

### FIELD

The present disclosure relates to methods for determining a source of wood products, wood preservative compositions, wood products, and methods of treating wood substrates.

### BACKGROUND

Certain wood products can be treated with wood preservatives and may have a label affixed thereto. The label can include identifying indicia, such as the name of the manufacturer of the treated wood product and the type of wood preservative used in the treating process. When the treated wood products are cut and/or used, the label may be separated from the treated wood product and/or might degrade excessively when subject to long term field exposure (e.g., ultraviolet radiation from the sun, moisture). Thus, identifying the source of the treated wood or the wood preservative used to treat to the wood product can be challenging.

### SUMMARY

The present disclosure provides a method for determining a source of a treated wood product. The method comprises detecting a presence of a marker in the treated wood product and determining the source of the treated wood product based on the detected presence of the marker.

The present disclosure also provides a wood preservative composition comprising at least 0.1% by weight of chromated copper arsenate (CCA) based on a total weight of the wood preservative composition, 0.01% to 10% by weight of a metal salt or a metal oxide based on the total weight of the wood preservative composition, and an aqueous solvent.

The present disclosure also provides a treated wood product produced by treating a wood substrate with a wood preservative composition according to the present disclosure.

The present disclosure also provides a method for manufacturing a treated wood product. The method comprises treating a wood substrate with a wood preservative composition according to the present disclosure.

It is understood that the inventions described in this specification are not limited to the examples summarized in this Summary. Various other aspects are described and exemplified herein.

### BRIEF DESCRIPTION OF DRAWINGS

The file of this patent contains at least one drawing/photograph executed in color. Copies of this patent with color drawing(s)/photograph(s) will be provided by the Office upon request and payment of the necessary fee.

The present invention will be more fully understood from the following detailed description and the accompanying drawings, which are not necessarily to scale, wherein:

FIG. 1 is a photograph showing a cross section of treated wood products treated with wood preservative composition D;

FIG. 2 is a photograph showing a cross section of treated wood products treated with wood preservative composition A:

FIG. 3 is a photograph showing a cross section of treated wood products treated with wood preservation composition B; and

FIG. 4 is a photograph showing a cross section of treated wood products treated with wood preservation composition C.

### DETAILED DESCRIPTION

Certain exemplary aspects of the present disclosure will now be described to provide an overall understanding of the principles of the structure, function, manufacture, and use of the compositions, methods, and products disclosed herein. One or more examples of these aspects are illustrated in the accompanying drawings. Those of ordinary skill in the art will understand that the devices and methods specifically described herein and illustrated in the accompanying drawings are non-limiting exemplary aspects and that the scope of the various examples of the present disclosure is defined solely by the claims. The features illustrated or described in connection with one exemplary aspect may be combined with the features of other aspects. Such modifications and variations are intended to be included within the scope of the present disclosure.

Any references herein to “examples,” “one example,” “an example,” similar references to “aspects,” or the like, means that a particular feature, structure, or characteristic described in connection with the example is included in at least one example. Thus, appearances of the phrases “examples,” “in one example,” “in an example,” similar references to “aspects,” or the like, in places throughout the specification are not necessarily all referring to the same example. Furthermore, the particular features, structures, or characteristics may be combined in any suitable manner in one or more examples. Thus, the particular features, structures, or characteristics illustrated or described in connection with one example may be combined, in whole or in part, with the features, structures, or characteristics of one or more other examples without limitation. Such modifications and variations are intended to be included within the scope of the present examples.

Chromated copper arsenate (CCA) can be used in a wood preservative composition for various purposes, including fungal decay control, antibacterial activity, and insecticidal activity. Visually identifying treated wood products that have been previously treated with CCA and exposed outdoor a few years has been difficult when a previously applied product label (e.g., end-tag) has been physically applied to, but separated from, the treated wood product. The present disclosure provides a method for determining a source of a treated wood product without the need for, or in the absence of, a physical label that can enable, for example, identification of the wood preservative composition used in the treatment of the wood product, the manufacturer of the wood preservative composition, and/or the manufacturer of the treated wood product.

The method for determining a source of a treated wood product according to the present disclosure comprises detecting a presence of a marker in the treated wood product and determining the source of the treated wood product based on the detected presence of a marker.

As used herein, the term “marker” is meant to mean any chemical composition that is employed either separately or as part of a wood preservative composition that serves as a reference point for a source of various manufacturing conditions, such as, for example, the identification of the wood preservative composition used in the treatment of the wood product, the manufacturer of the wood preservative composition, and/or the manufacturer of the treated wood product, as determined through various analytical identification techniques, such as chemical analysis, chemical detection, or visual observation. The marker as employed herein is not necessarily intended to provide chemical efficacy to the wood treatment composition; instead, it is meant to act solely as an identifier for various manufacturing conditions of the wood product.

The marker may be present in the wood product in various forms to provide identifiable effects in the wood chemistry of the wood product. For example, the marker may be present on an outer surface of the treated wood product and/or within pores of the treated wood product. The marker may be inert and/or provide minimal, if any, biocidal activity. The marker can be visually and/or analytically detected and may be resistant to decay in aqueous solutions and/or upon long term field exposure. The marker can comprise a metal oxide, a metal salt, or combinations thereof. The metal oxide and/or metal salt can comprise zinc, iron, or both. The metal salt can comprise a metal sulfate, a metal chloride, a metal nitrate, or combinations thereof. The metal oxide can comprise iron oxide, zinc oxide, or combinations thereof. For example, the marker can be selected based on a treatment administered to the wood product at a time of manufacture, a wood preservative composition used to treat the wood product, a manufacturer of the wood product, and/or a manufacturer of a wood preservative composition.

Detecting the presence of the marker can comprise determining a color of the treated wood product, measuring the presence of the marker with an analytical instrument, and/or contacting the treated wood product with a secondary indicator. In examples where detecting the presence of the marker comprises visually determining the color of the treated wood product, the color can correspond to the color of the marker. For example, a treated wood product comprising iron oxide can have a red, yellow, black and/or brown hue, or otherwise darkened appearance as a result of the presence of the marker in and/or on the treated wood product.

Contacting the treated wood product with a secondary indicator can provide a visual indication of the presence of the marker. For example, the secondary indicator may change color resulting from an interaction between the marker and the secondary indicator and the color change may be observed visually by a user and/or by a color reading device (e.g., spectrophotometer). The secondary indicator can comprise a chelating agent, such as, for example, a ligand that binds to the marker to produce an observable change in color responsive to interacting with the marker. The secondary indicator can comprise dithizone that may bind to zinc sulfate to form a pink colored complex.

The analytical instrument may be configured to detect the marker, such as, for example, a metal component of the marker (e.g., elemental zinc, elemental iron), a color of the marker, and/or the marker itself. For example, detecting the presence of the marker can comprise measuring the presence of the marker with an analytical instrument that utilizes X-ray fluorescence spectroscopy (XRF), atomic absorption spectroscopy (AAS), inductive coupled plasma spectroscopy (ICP), transmission electron microscopy optionally equipped with energy dispersive X-ray analysis (EDXA), scanning electron microscopy optionally equipped with energy dispersive X-ray analysis (EDXA), or combinations thereof. The analytical instrument can provide a quantitative measurement of the concentration of the marker in the wood product and/or a ratio of the concentration of the marker to a concentration of a second compound (e.g., a biocide). The analytical instrument may not utilize a visual assessment of the treated wood product and thus, may produce enhanced results when degradation and/or discoloration of treated wood product occurs after manufacture compared to a simple visual inspect.

Detecting the presence of the marker can be used to identify the treatment used to treat the wood product, the wood preservative composition used in the treatment, the manufacturer of the wood product, and/or the manufacturer of the wood preservative composition. The presence of the marker can be used to sort treated wood products that may otherwise have a substantially identical appearance.

Prior to detecting the presence of the marker, for example, a portion of the treated wood product may be removed to expose a surface of the treated wood product. Exposing a fresh surface of the treated wood product prior to detecting the presence of the marker can enable enhanced marker detection that may be due to removal of foreign materials from an outer surface of the treated wood product that were not present at the time of manufacture and/or leaching of the marker from an outer surface of the treated wood product. For example, foreign materials such as mold and dirt on an outer surface of the treated wood product may increase the difficulty of discerning a color of the wood product whereas an inner surface not previously exposed may be more representative of the wood product at the time of manufacture, including visual color and marker concentration. Removing a portion of the wood product to expose a surface of the wood product can comprise cutting, sawing, sanding, slicing, milling, machining, splitting, drilling, or combinations thereof.

The present disclosure also provides a method for manufacturing a treated wood product. The method comprises treating a wood substrate with a wood preservative composition, thereby forming a treated wood product. The wood preservative composition can comprise a biocide, the marker, and an aqueous solvent. The method can comprise, for example, contacting a wood substrate with the wood preservative composition for a period of time suitable to inhibit fungal decay of the wood substrate, thereby inhibiting biological deterioration (e.g., fungal decay) and insect attack of the wood product (e.g., termite attack). Contacting the wood substrate with the wood preservative composition can, for example, adhere the marker to an outer surface of the treated wood product and/or infiltrate the marker into a pore of the wood product.

Contacting the wood substrate can comprise a pressure process, a vacuum process, or combinations thereof. For example, the contacting can comprise a full-cell process, a modified full-cell process, an empty-cell process, or combinations thereof. Contacting the wood substrate can comprise the empty-cell process and the empty-cell process comprises a Rueping process and a Lowry process. For example, in the empty-cell process, prior to the introduction of wood preservative composition according to the present disclosure, the wood substrate is subjected to atmospheric air pressure (Lowry) or to higher air pressure than atmospheric air pressure (Rueping) of the necessary intensity and duration. Contacting processes that can be used with the present disclosure are defined in the American Wood Protection Association (AWPA) Book of Standards T1-21: Processing and Treatment Standard, which is hereby incorporated by reference. As used herein, atmospheric air pressure refers to 1 atmosphere absolute.

The contacting can be performed until the wood product retains at least 0.1 pounds biocide actives per cubic food (pcf) of the wood product, such as, for example, at least 0.15 pounds of biocide actives pcf of the wood product. In examples where the wood preservative composition comprises CCA, the contacting can be performed until the wood product retains at least 0.1 pounds CCA actives pcf of the wood product, such as, for example, at least 0.15 pounds of CCA actives pcf of the wood product. CCA actives are described in AWPA Book of Standards P23, which is hereby incorporate by reference.

The contacting can be performed until the wood product retains at least 10 grams of the marker per cubic meter of the treated wood product, such as, for example, at least 15 grams, or at least 20 grams of the marker per cubic meter of the treated wood product. For example, the contacting can be performed until the wood product retains 10 grams to 1 kg of marker per cubic meter of the treated wood product. The contacting can be performed until, for example, the wood product retains at least 10 grams of metal salt and/or metal oxide per cubic meter of the treated wood product, such as, for example, at least 15 grams, or at least 20 grams of the metal salt and/or metal oxide all per cubic meter of the treated wood product. The contacting can be performed until the wood product retains 10 grams to 1 kg of metal salt and/or metal oxide per cubic meter of the treated wood product.

As provided here, the wood preservative composition can comprise a biocide, the marker, and an aqueous solvent.

The concentration of the biocide in the wood preservative composition can be selected based on the desired level of inhibition of the fungal decay, desired level of inhibition of insect attack, the method used to treat the wood product, and/or the desired storage and/or shipping conditions. The biocide can comprise CCA. CCA can comprise a mixture of chromium, copper, and arsenic compounds that can inhibit fungal decay and/or insect attack of the treated wood product. For example, CCA can comprise hexavalent chromium compounds (e.g., potassium dichromate, sodium dichromate, and/or chromium trioxide), bivalent copper compounds (e.g., copper sulfate, basic copper carbonate, and/or cupric oxide or hydroxide), and pentavalent arsenic compounds (e.g., arsenic pentoxide, arsenic acid, sodium arsenate, and/or pyroarsenate). The CCA of the wood preservative composition can comprise hexavalent chromium compounds in a range of 43.0% by weight to 51.0% by weight, bivalent copper compounds in a range of 12.0% by weight to 21.0% by weight, and pentavalent arsenic compounds in a range of 30.0% by weight to 43.0% by weight, all based on the total weight of the CCA. The CCA of the wood preservative composition can comprise oxides of hexavalent chromium, bivalent copper, and pentavalent arsenic. For example, the CCA can comprise chromium trioxide (CrO₃) in a range of 43.0% by weight to 51.0% by weight, cupric oxide (CuO) in a range of 12.0% by weight to 21.0% by weight, and arsenic pentoxide (As₂O₅) in a range of 30.0% by weight to 43.0% by weight, all based on the total weight of the CCA. CCA can comprise 47.5% by weight CrO₃, 18.5% by weight CuO, and 34.0% As₂O₅, all based on the total weight of the CCA.

The wood preservative composition can comprise a concentration of CCA of at least 0.1% by weight based on the total weight of the composition, such as, for example, at least 0.5% by weight, at least 0.6% by weight, at least 1% by weight, at least 2% by weight, at least 3% by weight, at least 4% by weight, or at least 5% by weight, all based on the total weight of the wood preservative composition. The wood preservative composition can comprise a concentration of CCA no greater than 10% by weight based on the total weight of the composition, such as, for example, no greater than 9% by weight, no greater than 8% by weight, no greater than 6% by weight, no greater than 5% by weight, no greater than 4% by weight, no greater than 3% by weight, or no greater than 2% by weight, all based on the total weight of the wood preservative composition. The wood preservative composition can comprise a concentration of CCA in a range of 0.1% by weight to 10% by weight based on the total weight of the composition, such as, for example, 0.6% by weight to 6% by weight based on the total weight of the composition. For example, the wood preservative composition can comprise 1.8% by weight CCA based on the total weight of the composition.

The wood preservative composition can comprise a concentration of the marker of at least 0.01% by weight based on the total weight of the composition, such as, for example, at least 0.02% by weight, at least 0.03% by weight, at least 0.04% by weight, at least 0.05% by weight, or at least 1.0% by weight, all based on the total weight of the wood preservative composition. The wood preservative composition can comprise a concentration of the marker of no greater than 10% by weight based on the total weight of the wood preservative composition, such as, for example, no greater than 9% by weight, no greater than 8% by weight, no greater than 6% by weight, no greater than 5% by weight, no greater than 4% by weight, or no greater than 3% by weight, of the marker all based on the total weight of the wood preservative composition. The wood preservative composition can comprise 0.01% by weight to 10% by weight of a marker based on the total weight of the composition, such as, for example, 0.01% by weight to 5.0% by weight, 0.1% by weight to 3.0% by weight of the marker, 0.01% by weight to 1.0% by weight, 0.01% by weight to 0.9% by weight, 0.05% by weight to 0.9% by weight, or 0.1% by weight to 0.8% by weight, of the marker all based on the total weight of the wood preservative composition. For example, the wood preservative composition can comprise 0.01% by weight to 10% by weight of a metal salt and/or metal oxide based on the total weight of the composition, such as, for example, 0.01% by weight to 5.0% by weight, 0.1% by weight to 3.0% by weight of the marker, 0.01% by weight to 1.0% by weight, 0.01% by weight to 0.9% by weight, 0.05% by weight to 0.9% by weight, or 0.1% by weight to 0.8% by weight, of a metal salt and/or metal oxide all based on the total weight of the composition. For example, the wood preservative composition according to the present disclosure can comprise 0.2% by weight zinc sulfate based on the total weight of the composition.

A weight ratio of the biocide to the marker in the wood preservative composition can be in a range of 1:10 to 500:1, such as, for example, 1:1 to 500:1, 1:1 to 200:1, or 2:1 to 200:1. For example, a weight ratio of CCA to the a metal salt and/or metal oxide in the wood preservative composition can be in a range of 1:10 to 500:1, such as, for example, 1:1 to 500:1, 1:1 to 200:1, or 2:1 to 200:1.

The marker can be, but is not necessarily, present in a dispersed phase of the wood preservative composition. When incorporated into the wood preservative composition, for example, the marker compound can comprise iron oxide and/or zinc sulfate particles, which may be combined with an aqueous dispersion of a CCA-based composition to form the wood preservative composition.

The wood preservative composition can comprise a concentration of the aqueous solvent (e.g., water) of at least 60% by weight, such as, for example, at least 70% by weight, at least 80% by weight, at least 85% by weight, at least 90% by weight, or at least 95% by weight all based on the total weight of the wood preservative composition. The wood preservative composition can comprise a concentration of the aqueous solvent of no greater than 99% by weight based on the total weight of the wood preservative composition, such as, for example, no greater than 98%, no greater than 95%, or no greater than 90% by weight of the aqueous solvent based on the total weight of the wood preservative composition. The wood preservative composition can comprise a concentration of aqueous solvent in a range of 60% to 99% by weight based on the total weight of the wood preservative composition, such as, for example, 80% to 99% by weight, 85% to 99% by weight, or 95% to 99% by weight aqueous solvent all based on the total weight of the wood preservative composition.

The wood preservative composition can comprise an optional secondary solvent. The secondary solvent can comprise a polar protic solvent and/or a polar aprotic solvent. A polar protic solvent contains an OH group and/or an NH group that is able to form hydrogen bonds. Polar protic solvents are highly polar because of the OH group and/or NH group. For example, the polar protic solvent can have a dielectric constant greater than 15. The polar aprotic solvent has a medium range of polarity. The polar aprotic solvent has a polar bonds of C═O, S═O, or the like, which typically have a polarity less than that of an OH group and an NH group. For example, the polar aprotic solvent has an dielectric constant in a range of 5 to 15.

A polar protic solvent containing an OH group can comprise amyl alcohol, benzyl alcohol, cyclohexanol, ethyl alcohol-denatured, 2-ethyl hexanol, Exxal 8® isooctyl alcohol, Exxal 10® isodecyl alcohol, Exxal 13® Tridecyl Alcohol, furfuryl alcohol, isobutyl alcohol, isopropyl alcohol 99% anhy, methanol, methyl amyl alcohol (MIBC), n-butyl alcohol, n-propyl alcohol, Neodol® Linear Alcohol, secondary butyl alcohol, tertiary butyl alcohol, tetrahydrofurfuryl alcohol, Texanol Ester Alcohol®, UCAR Filmer IBT®, diethylene glycol, dipropylene glycol, ethylene glycol, glycerine 96%, 99%, U.S.P., glycerine, hexylene glycol, Neol® neopentyiglycol, polyethylene glycol, polypropylene glycol, Propylene Glycol Ind., U.S.P., tetraethylene glycol, triethylene glycol, tripropylene glycol, or combinations thereof. The polar organic solvent can comprise benzyl alcohol.

A polar protic solvent containing an NH group can comprise diamylamine, diethylamine, diisopropylamine, dimethylethylamine, di-n-butylamine, mono-2-ethylhexyamine, monoamylamine, monoethylamine 70%, monoisopropylamine, anhy., mono-n-butylamine, triamylamine, triethylamine, tri-n-butylamine, dibutylaminoethanol, diethylaminoethanol, diethylaminoethoxyethanol, diisopropylaminoethanol, dimethylamino-2P, 77% mixed, simethylamino-2-P, anhy., dimethylaminoethanol, dimethylaminoethoxyethanol, ethylaminoethanol, ethylaminoethanol, mixed, isopropylaminoethanol, isopropylaminoethanol, mixed, methyldiethanolamine, monomethylaminoethanol, mono-n-propylaminoethanol, n-butylaminoethanol, n-butyldiethanolamine, n-butyldiethanolamine, photo, t-butylaminoethanol, t-butyldiethanolamine, diethanolamine, donoethanolamine, triethanolamine, triethanolamine 85%/99%, diisopropanolamine, monoisopropanolamine, triisopropanolamine, aminoethylethanolamine, aminoethylpiperazine, diethylenetriamine, ethylenediamine, piperazine 65%/anhy., piperazine, tetraethylenepentamine, triethylenetetramine, 3-methoxypropylamine, AMP® Regular/95, cyclohexylamine, morpholine, Neutrol TE®, or combinations thereof.

The polar aprotic solvent can comprise acetone, cyclohexanone, diacetone, diisobutyl ketone (DIBK), isophorone, methyl amyl ketone (MAK), methyl ethyl ketone (MEK), methyl isoamyl ketone (MIAK), methyl isobutyl ketone (MIAK), methyl propyl ketone (MPK), methylene chloride, monochlorobenzene, orthodichlorobenzene, perchloroethylene, trichloroethylene, Vertrel® Hydrofluorocarbon, amyl acetate, dibasic ester, ethyl acetate, 2 ethyl hexyl acetate, ethyl propionate, Exxate® acetate esters, isobutyl acetate, isobutyl isobuterate, isopropyl acetate, n-butyl acetate, n-butyl propionate, n-pentyl propionate, n-propyl acetate, dimethyl sulfoxide (DMSO), dimethylformamide (DMF), tetrahydrofuran (THF), or combinations thereof.

The wood preservative composition according to the present disclosure can comprise one or more additional additives. For examples, the wood preservative composition according to the present disclosure can also optionally comprise a colorant, an ultraviolet (UV) stabilizer, a UV absorber, de-foamer, a water repellent, an additional biocide, a fungicide, a termiticide, a fire retardant, a mold inhibitor, a wax oil emulsion, an additional solvent, or combinations thereof. The UV stabilizer can comprise benzophenone, substituted benzophenones, cinnamic acid, esters of cinnamic acid, amides of cinnamic acid, substituted triazines (e.g., triphenyl triazaine, substituted phenyl triazine), or combinations thereof. The UV absorber can comprise benzotriazole, substituted benzotriazole, hindered amine light stabilizers, or combinations thereof. The water repellent can comprise a wax water repellent (e.g., paraffin wax, polyethylene wax, carnauba wax, slack wax), a silicone, or combinations thereof.

The fire retardant can be one or more compounds selected from the group consisting of inorganic metal oxides, hydroxides, salts and expandable graphite phosphate compounds, nitrogen-containing compounds, dipentaerythritol, pentaerythritol, dextrin and boron-containing compounds.

The additional biocide can be used in addition to CCA or in place of CCA. The additional biocide can comprise a creosote, a triazole, an imidazole, a pyrazole, a boron compound, a quaternary ammonium, an isothiazolone, a pyrethroid, copper metal, a copper compound (e.g., copper napthenate), pentachlorophenol, bethoxazin, or combinations thereof.

Triazole and imidazole can comprise: 1-[[2-(2,4-dichlorophenyl)-1,3-dioxolan-2-yl]methyl]-1H-1,2,4-triazole (azaconazole), 1-[(2RS,4RS: 2RS,4SR)-4-bromo-2-(2,4-dichlorophenyl)tetrahydrofurfuryl]-1H-1,2,4-triazole (bromuconazole), (2RS,3RS: 2RS,3SR)-2-(4-chlorophenyl)-3-cyclopropyl-1-(1H-1,2,4-triazol-1-yl) butan-2-ol (Cyproconazole), (2RS,3RS)-1-(2,4-dichlorophenyl)-4,4-dimethyl-2-(1H-1,2,4-triazol-1-yl) pentan-3-ol (diclobutrazol), cis-trans-3-chloro-4-[4-methyl-2-(1H-1,2,4-triazol-1-ylmethyl)-1,3-dioxolan-2-yl] phenyl 4-chlorophenyl ether (difenoconazole), (E)-(RS)-1-(2,4-dichlorophenyl)-4,4-dimethyl-2-(1H-1,2,4-triazol-1-yl) pent-1-en-3-ol (diniconazole), (E)-(R)-1-(2,4-dichlorophenyl)-4,4-dimethyl-2-(1H-1,2,4-triazol-1-yl) pent-1-en-3-ol (diniconazole-M), (2RS,3SR)-1-[3-(2-chlorophenyl)-2,3-epoxy-2-(4-fluorophenyl) propyl]-1H-1,2,4-triazole (epoxiconazole), (RS)-1-[2-(2,4-dichlorophenyl)-4-ethyl-1,3-dioxolan-2-ylmethyl]-1H-1,2,4-triazole (etaconazole), (RS)-4-(4-chlorophenyl)-2-phenyl-2-(1H-1,2,4-triazol-1-ylmethyl) butyronitrile (fenbuconazole), 3-(2,4-dichlorophenyl)-6-fluoro-2-(1H-1,2,4-triazol-1-yl) quinazolin-4 (3H)-one (fluquinconazole), bis(4-fluorophenyl)(methyl)(1H-1,2,4-triazol-1-ylmethyl) silane (flusilazole), (RS)-2,4′-difluoro-α-(1H-1,2,4-triazol-1-ylmethyl)benzhydryl alcohol (flutriafol), (2RS,5RS: 2RS,5SR)-5-(2,4-dichlorophenyl)tetrahydro-5-(1H-1,2,4-triazol-1-ylmethyl)-2-furyl 2,2,2-trifluoroethyl ether (furconazole), (2RS,5RS)-5-(2,4-dichlorophenyl)tetrahydro-5-(1H-1,2,4-triazol-1-ylmethyl)-2-furyl 2,2,2-trifluoroethyl ether (furconazole-cis), (RS)-2-(2,4-dichlorophenyl)-1-(1H-1,2,4-triazol-1-yl) hexan-2-ol (hexaconazole), 4-chlorobenzyl (EZ)—N-(2,4-dichlorophenyl)-2-(1H-1,2,4-triazol-1-yl)thioacetamidate (imibenconazole), (1RS,2SR,5RS;1RS,2SR,5SR)-2-(4-chlorobenzyl)-5-isopropyl-1-(1H-1,2,4-triazol-1-ylmethyl)cyclopentanol (ipconazole), (1RS,5RS;1RS,5SR)-5-(4-chlorobenzyl)-2,2-dimethyl-1-(1H-1,2,4-triazol-1-ylmethyl)cyclopentanol (metconazole), (RS)-2-(4-chlorophenyl)-2-(1H-1,2,4-triazol-1-ylmethyl) hexanenitrile (myclobutanil), (RS)-1-(2,4-dichloro-β-propylphenethyl)-1H-1,2,4-triazole (penconazole), cis-trans-1-[2-(2,4-dichlorophenyl)-4-propyl-1,3-dioxolan-2-ylmethyl]-1H-1,2,4-triazole (propiconazole), (RS)-2-[2-(1-chlorocyclopropyl)-3-(2-chlorophenyl)-2-hydroxypropyl]-2,4-dihydro-1,2,4-triazole-3-thione (prothioconazole), 3-(2,4-dichlorophenyl)-2-(1H-1,2,4-triazol-1-yl)-quinazolin-4 (3H)-one (quinconazole), (RS)-2-(4-fluorophenyl)-1-(1H-1,2,4-triazol-1-yl)-3-(trimethylsilyl) propan-2-ol (simeconazole), (RS)-1-p-chlorophenyl-4,4-dimethyl-3-(1H-1,2,4-triazol-1-ylmethyl) pentan-3-ol (tebuconazole), propiconazole, (RS)-2-(2,4-dichlorophenyl)-3-(1H-1,2,4-triazol-1-yl) propyl 1,1,2,2-tetrafluoroethyl ether (tetraconazole), (RS)-1-(4-chlorophenoxy)-3,3-dimethyl-1-(1H-1,2,4-triazol-1-yl) butan-2-one (triadimefon), (1RS,2RS: 1RS,2SR)-1-(4-chlorophenoxy)-3,3-dimethyl-1-(1H-1,2,4-triazol-1-yl) butan-2-ol (triadimenol), (RS)-(E)-5-(4-chlorobenzylidene)-2,2-dimethyl-1-(1H-1,2,4-triazol-1-ylmethyl)cyclopentanol (triticonazole), (E)-(RS)-1-(4-chlorophenyl)-4,4-dimethyl-2-(1H-1,2,4-triazol-1-yl) pent-1-en-3-ol (uniconazole), (E)-(S)-1-(4-chlorophenyl)-4,4-dimethyl-2-(1H-1,2,4-triazol-1-yl) pent-1-en-3-ol (uniconazole-P), 2-(2,4-difluorophenyl)-1-(1H-1,2,4-triazole-1-yl)-3-trimethylsilyl-2-propanol, or combinations thereof. Other azole compounds suitable as an additional biocide can comprise amisulbrom, bitertanol, fluotrimazole, triazbutil, climbazole, clotrimazole, imazalil, oxpoconazole, prochloraz, triflumizole, azaconazole, simeconazole, hexaconazole, or combinations thereof.

The pyrazole can comprise: benzovindiflupyr, bixafen, fenpyrazamine, fluxapyroxad, furametpyr, isopyrazam, oxathiapiprolin, penflufen, penthiopyrad, pydiflumetofen, pyraclostrobin, pyrametostrobin, pyraoxystrobin, rabenzazole, sedaxane, or combinations thereof.

The boron compound can comprise water-insoluble boron compounds, such as, for example, metal borate compounds (e.g., calcium borate, borate silicate, aluminum silicate borate hydroxide, silicate borate hydroxide fluoride, hydroxide silicate borate, sodium silicate borate, calcium silicate borate, aluminum borate, boron oxide, magnesium borate, iron borate, copper borate, zinc borate (borax)), or combinations thereof.

The quaternary ammonium can comprise didecyldimethylammonium chloride; didecyldimethylammonium carbonate/bicarbonate: alkyldimethylbenzylammonium chloride; alkyldimethylbenzylammonium carbonate/bicarbonate: didodecyldimethylammonium chloride; didodecyldimethylammonium carbonate/bicarbonate: didodecyldimethylammonium propionate; N, N-didecyl-N-methyl-poly(oxyethyl)ammonium propionate, or combinations thereof.

The isothiazolone can comprise methylisothiazolinone: 5-chloro-2-methyl-4-isothiazoline-3-one, 2-methyl-4-isothiazoline-3-one, 2-n-octyl-4-isothiazoline-3-one, 4,5-dichloro-2-n-octyl-4-isothiazoline-3-one, 2-ethyl-4-isothiazoline-3-one, 4,5-dichloro-2-cyclohexyl-4-isothiazoline-3-one, 5-chloro-2-ethyl-4-isothiazoline-3-one, 2-octyl-3-isothiazolone, 5-chloro-2-t-octyl-4-isothiazoline-3-one, 1,2-benzisothiazoline-3-one, preferably 5-chloro-2-methyl-4-isothiazoline-3-one, 2-methyl-4-isothiazoline-3-one, 2-n-octyl-4-isothiazoline-3-one, 4,5-dichloro-2-n-octyl-4-isothiazoline-3-one, 1,2-benzisothiazoline-3-one, etc., more preferably 5-chloro-2-methyl-4-isothiazoline-3-one, 2-n-octyl-4-isothiazoline-3-one, 4,5-dichloro-2-n-octyl-4-isothiazoline-3-one, 1,2-benzisothiazoline-3-one, chloromethylisothiazolinone, 4,5-Dichloro-2-n-octyl-3 (2H)-isothiazolone, 1,2-benzisothiazolin-3-one, or combinations thereof.

The pyrethroid can comprise: acrinathrin, allethrin, bioallethrin, barthrin, bifenthrin, bioethanomethrin, cyclethrin, cycloprothrin, cyfluthrin, beta-cyfluthrin, cyhalothrin, gamma-cyhalothrin, lambda-cyhalothrin, cypermethrin, alpha-cypermethrin, beta-cypermethrin, theta-cypermethrin, zeta-cypermethrin, cyphenothrin, deltamethrin, dimefluthrin, dimethrin, empenthrin, fenfluthrin, fenpirithrin, fenpropathrin, fenvalerate, esfenvalerate, flucythrinate, fluvalinate, tau-fluvalinate, furethrin, imiprothrin, metofluthrin, permethrin, biopermethrin, transpermethrin, phenothrin, prallethrin, profluthrin, pyresmethrin, resmethrin, bioresmethrin, cismethrin, tefluthrin, terallethrin, tetramethrin, tralomethrin, transfluthrin, etofenprox, flufenprox, halfenprox, protrifenbute, silafluofen, or combinations thereof.

The additional biocide can comprise imidachloprid, fipronil, cyfluthrin, bifenthrin, permethrin, cypermethrin, chlorpyrifos, iodopropynyl butylcarbamate (IPBC), chlorothalonil, 2-(thiocyanatomethylthio)benzothiazole, alkoxylated diamines, carbendazim, or combinations thereof. The additional biocide can comprise a bactericide, a moldicide, or combinations thereof.

The wood preservative composition according to the present disclosure can be a concentrated composition or a diluted treating composition. For example, the concentrated composition described herein can be diluted with another solvent, which may be one or more of the same or different aqueous solvent used to formulate the concentrated composition, to form the diluted treating composition. For example, the diluted treating composition may be directly formed without first forming the concentrated composition. In addition, and for example, the concentrated composition may be produced as a liquid. In addition, the resulting concentrate can be further diluted in another solvent to make the final wood preservative treating composition. For example, the concentrated composition may be used to treat a wood substrate to form a wood product.

The wood preservative composition can exhibit a pH in a range of 0.1 to 3.0 as measured at a temperature in a range of 20° C. to 30° C. In examples where the wood preservative composition was prepared by diluting a concentrated composition, the wood preservative composition may further comprise a pH adjustment agent (e.g., acid, base) to adjust the pH of the diluted composition.

The treated wood product can comprise timber, plywood, laminated veneer lumber (LVL), cross laminated timber (CTL), parallel strand lumber (PSL), structural glued laminated timber, particle board, dimensional lumber, or combinations thereof. For example, the treated wood product can comprise a deck, a rail, a fence, a utility pole, a post, a pile, a railway tie, a railroad bridge, cladding, siding, or combinations thereof.

The treated wood product can comprise various species of wood. For example, the treated wood product can comprise eucalyptus wood (e.g., eucalyptus planted in Brazil, Chile, Argentina, Uruguay, New Zealand, Australia, other South American Countries, Central America: Corymbia, Eucalyptus pilularis), southern pine, Douglas fir, Jack pine, red pine, Lodgepole pine, radiata pine (e.g., radiata pine grown in Brazil, Chile, Argentina, Uruguay, New Zealand, Australia, other South American Countries, Central America: P. elliotti, P. caribaea, P. patula), Alaska yellow cedar, Hem-fir, Nordic pine, Scotts pine, white spruce, Spruce-Pine-Fir, redwood, white oak, red oak, maple, black and red gum, Norway spruce, Sitka spruce, western red cedar, western larch, ponderosa pine, or combinations thereof.

In examples where the treated wood product is formed by treating a wood substrate with a wood preservative composition comprising oxides of hexavalent chromium, bivalent copper and pentavalent arsenic, the wood product can retain at least 0.1 pounds per cubic foot (pcf) of total CCA active oxides as defined in 2023 AWPA Book of Standards. For example, the treated wood product can be produced by contacting a wood substrate with the wood preservative composition according to the present disclosure for a period of time suitable to inhibit fungal decay of the wood substrate, such that the wood product retains at least 0.15 pounds pcf of total CCA active oxides as defined in 2023 AWPA Book of Standards. Furthermore, the treated wood product can comprise a metal salt and/or a metal oxide such that the metal salt and/or metal oxide exhibits a leachability of no greater than 40% from the wood product measured according to AWPA E11 leaching water study.

The present disclosure will be more fully understood by reference to the following examples, which provide illustrative non-limiting aspects of the invention. It is understood that the invention described in this specification is not necessarily limited to the examples described in this section.

### Penetration Testing

2 inch×6 inch×48 inch long wood substrates comprising southern pine were pressure treated using wood preservative compositions A-D to form treated wood products. Wood preservative compositions A-D comprise CCA, various markers, and an aqueous solvent. The composition of wood preservative compositions A-D is shown in Table 1 below.

TABLE 1
Wood Preservative Compositions

| | CCA | Iron Oxide | | |
|---|---|---|---|---|
| | Concen- | Marker | Zinc Sulfate | |
| Wood | tration | Concen- | Concen- | |
| Preservative | (% actives | tration | tration | Aqueous |
| Composition | by weight) | (% by weight) | (% by weight) | Solvent |
| A | 1.80 | | 0.02 | Balance |
| B | 1.80 | | 0.04 | Balance |
| C | 1.80 | | 0.08 | Balance |
| D | 1.80 | 0.25 | | Balance |

The wood substrates were treated with full cell treatment cycles. Each wood substrate was weighed before and after treatment to determine the CCA and marker retention in the treated wood products. After pressure treatment, the treated wood product was air dried.

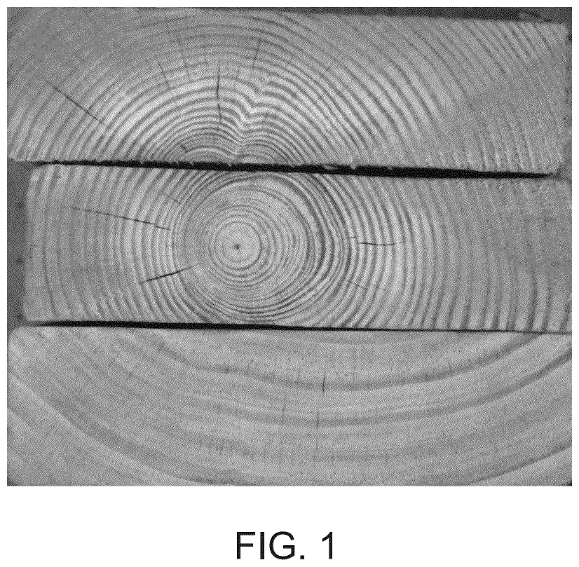

Afterward, cross-sections were cut out of the treated wood products to be further evaluated for penetration and detectability of the marker. Penetration of the iron oxide was visually observed as darker areas compared to untreated areas in cross-sections of treated wood products treated with wood preservative composition D as shown in FIG. 1. As illustrated, rings of darker treated areas are separated by untreated rings having a natural wood appearance. Penetration of cross-sections of treated wood products treated with wood preservative compositions A-C were evaluated by contacting (e.g., spraying) the samples with a secondary indicator solution of dithizone dissolved in an ethanol solvent at 0.1% weight/volume.

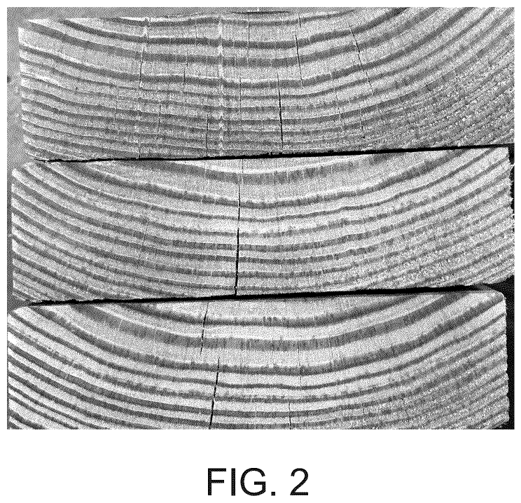

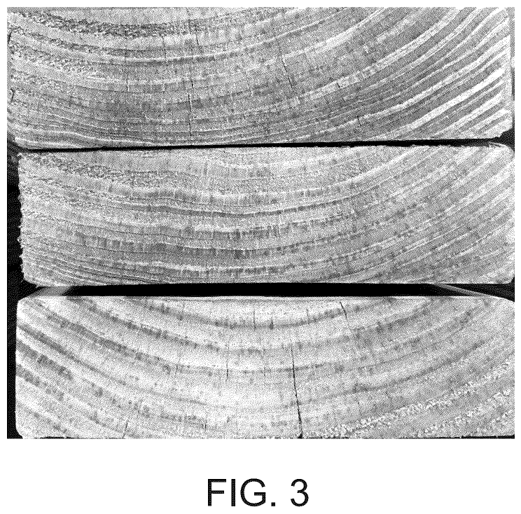

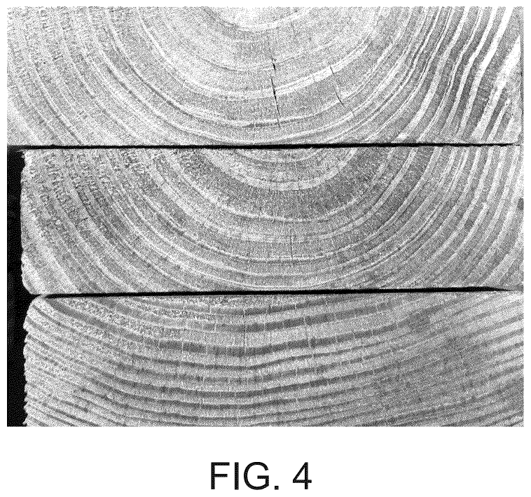

The results of cross-sections of treated wood products treated with wood preservative composition A is shown in FIG. 2. The results of cross-sections of treated wood products treated with wood preservative composition B is shown in FIG. 3. The results of cross-sections of treated wood products treated with wood preservative composition C is shown in FIG. 4. As illustrated in FIGS. 2-4, the cross-section has a pink appearance after being exposed to the secondary indicator. The pink color indicated the presence of zinc. Increasing the loading of the zinc sulfate in wood substrate increases the intensity of the color response from the applied indicator.

Table 2 shows the retention results for the wood substrates treated with wood preservative composition A. Table 3 shows retention results for the wood substrates treated with the wood preservative composition B. Table 4 shows retention results for the wood substrates treated with the wood preservative composition C. Table 5 shows retention results for the wood substrates treated with the wood preservative composition D.

TABLE 2
Retention Results of 2-inch × 6-inch Wood Substrates
Treated with Wood Preservative Composition A

| | | | | Wood | | |
|---|---|---|---|---|---|---|
| | | | | Preservative | Zinc | |
| | | | Volume of | Composition | Sulfate in | CCA in |
| Wood | Before | After | Sample | in Sample | Sample | Sample |
| Substrate | (lbs) | (lbs) | (Ft³) | (pcf) | (pcf) | (pcf) |
| 1-1 | 9.13 | 17.09 | 0.2292 | 34.73 | 0.069 | 0.6252 |
| 1-2 | 9.18 | 18.03 | 0.2292 | 38.62 | 0.0077 | 0.6951 |
| 1-3 | 8.87 | 17.89 | 0.2292 | 39.36 | 0.0079 | 0.7085 |
| Average of | — | — | — | — | 0.0075 | 0.6763 |
| Wood | | | | | | |
| Substrates | | | | | | |

TABLE 3
Retention Results of 2-inch × 6-inch Wood Substrates
Treated with Wood Preservative Composition B

| | | | | Wood | | |
|---|---|---|---|---|---|---|
| | | | | Preservative | Zinc | |
| | | | Volume of | Composition | Sulfate in | CCA in |
| Wood | Before | After | Sample | in Sample | Sample | Sample |
| Substrate | (lbs) | (lbs) | (Ft³) | (pcf) | (pcf) | (pcf) |
| 2-1 | 9.12 | 18.37 | 0.2292 | 40.36 | 0.0163 | 0.7265 |
| 2-2 | 7.77 | 17.54 | 0.2292 | 42.63 | 0.0173 | 0.7674 |
| 2-3 | 7.80 | 17.63 | 0.2292 | 42.89 | 0.0174 | 0.7721 |
| Average of | — | — | — | — | 0.0170 | 0.7553 |
| Wood | | | | | | |
| Substrates | | | | | | |

TABLE 4
Retention Results of 2-inch × 6-inch Wood Substrates
Treated with Wood Preservative Composition C

| | | | | Wood | | |
|---|---|---|---|---|---|---|
| | | | | Preservative | Zinc | |
| | | | Volume of | Composition | Sulfate in | CCA in |
| Wood | Before | After | Sample | in Sample | Sample | Sample |
| Substrate | (lbs) | (lbs) | (Ft³) | (pcf) | (pcf) | (pcf) |
| 3-1 | 9.19 | 18.35 | 0.2292 | 39.97 | 0.0324 | 0.7195 |
| 3-2 | 7.26 | 16.48 | 0.2292 | 40.23 | 0.0326 | 0.7242 |
| 3-3 | 7.16 | 16.48 | 0.2292 | 40.67 | 0.0329 | 0.7320 |
| Average of | — | — | — | — | 0.0326 | 0.7252 |
| Wood | | | | | | |
| Substrates | | | | | | |

TABLE 5
Retention Results of 2-inch × 6-inch Wood Substrates
Treated with Wood Preservative Composition D

| | | | | Wood | | |
|---|---|---|---|---|---|---|
| | | | | Preservative | | |
| | | | Volume of | Composition | Iron Oxide | CCA in |
| Wood | Before | After | Sample | in Sample | in Sample | Sample |
| Substrate | (lbs) | (lbs) | (Ft³) | (pcf) | (pcf) | (pcf) |
| 4-1 | 9.86 | 16.69 | 0.2292 | 29.80 | 0.0745 | 0.5365 |
| 4-2 | 10.29 | 16.31 | 0.2292 | 26.27 | 0.0657 | 0.4728 |
| 4-3 | 9.10 | 18.35 | 0.2292 | 40.36 | 0.1009 | 0.7265 |
| Average of | — | — | — | — | 0.0804 | 0.5786 |
| Wood | | | | | | |
| Substrates | | | | | | |

### Leaching Analysis

¾-inch wood substrates suitable for AWPA E11 leaching testing were treated with CCA based wood preservative compositions according to the present disclosure. For example, A first set of the wood substrates was treated with wood preservative composition A. A second set of the wood substrates was treated with wood preservative composition D. Both sets were treated with full cell treatment cycles. Each wood substrate was weighed before and after treatment to determine the CCA retention in the treated wood products. The twelve treated wood products closest to the target retention of 0.60 pcf CCA were chosen and evenly separated into two further sets where the retentions of CCA were matched.

An AWPA E11 leaching water study was also conducted to determine if zinc sulfate and iron oxide based markers would remain in the treated wood product at measurable levels. One treated wood product in the sets was leached and a second wood product remained unleached. After leaching both sets were dried, ground and analyzed by ICP with the iron oxide treated blocks tested for iron (Fe) and the zinc sulfate treated blocks tested for zinc (Zn). Table 6 shows the retention results for the ¾-inch wood substrates treated with the wood preservative comprising iron oxide. Table 7 shows retention results for the ¾-inch wood substrates treated with the wood preservative comprising zinc sulfate.

TABLE 6
Retention Results of ¾-inch AWPA E11 Wood
Substrates treated with Wood Preservative Composition A

| | | | | Wood | | | |
|---|---|---|---|---|---|---|---|
| | | | | Preservative | | | |
| | | | Volume of | Composition | Iron oxide | CCA in | |
| Wood | Before | After | Sample | in Sample | in Sample | Sample | |
| Substrate | (lbs) | (lbs) | (Ft³) | (pcf) | (pcf) | (pcf) | State |
| B20 | 3.81 | 8.29 | 0.000244 | 40.5 | 0.101 | 0.566 | Leached |
| B3 | 3.81 | 8.45 | 0.000244 | 41.9 | 0.105 | 0.587 | Unleached |
| B5 | 3.88 | 8.54 | 0.000244 | 42.1 | 0.105 | 0.589 | Leached |
| B2 | 3.53 | 8.24 | 0.000244 | 42.5 | 0.106 | 0.595 | Unleached |
| B12 | 3.57 | 8.34 | 0.000244 | 43.1 | 0.108 | 0.603 | Leached |
| B4 | 3.7 | 8.49 | 0.000244 | 43.3 | 0.108 | 0.606 | Unleached |
| B10 | 3.33 | 8.15 | 0.000244 | 43.5 | 0.109 | 0.609 | Leached |
| B19 | 3.32 | 8.14 | 0.000244 | 43.5 | 0.109 | 0.609 | Unleached |
| B11 | 3.25 | 8.09 | 0.000244 | 43.7 | 0.109 | 0.612 | Leached |
| B16 | 3.5 | 8.36 | 0.000244 | 43.9 | 0.110 | 0.614 | Unleached |
| B18 | 3.43 | 8.3 | 0.000244 | 44.0 | 0.110 | 0.616 | Leached |
| B17 | 3.61 | 8.5 | 0.000244 | 44.2 | 0.111 | 0.618 | Unleached |
| Average of | — | — | — | — | 0.107 | 0.599 | Leached |
| Leached Wood | | | | | | | |
| Substrates | | | | | | | |
| Average of | — | — | — | — | 0.108 | 0.605 | Unleached |
| Unleached | | | | | | | |
| Wood | | | | | | | |
| Substrates | | | | | | | |

TABLE 7
Retention Results of ¾-inch AWPA E11Wood
Substrates treated with Wood Preservative Composition D

| | | | | Wood | | | |
|---|---|---|---|---|---|---|---|
| | | | | Preservative | Zinc | | |
| | | | Volume of | Composition | Sulfate in | CCA in | |
| Wood | Before | After | Sample | in Sample | Sample | Sample | Sample |
| Substrate | (lbs) | (lbs) | (Ft³) | (pcf) | (pcf) | (pcf) | Set |
| A1 | 3.48 | 8.25 | 0.000244 | 43.1 | 0.035 | 0.603 | Leached |
| A3 | 3.77 | 8.54 | 0.000244 | 43.1 | 0.035 | 0.603 | Unleached |
| A2 | 3.21 | 8.02 | 0.000244 | 43.4 | 0.035 | 0.608 | Leached |
| A13 | 3.53 | 8.34 | 0.000244 | 43.4 | 0.035 | 0.608 | Unleached |
| A4 | 3.34 | 8.16 | 0.000244 | 43.5 | 0.035 | 0.609 | Leached |
| A18 | 3.77 | 8.6 | 0.000244 | 43.6 | 0.035 | 0.611 | Unleached |
| A17 | 3.69 | 8.53 | 0.000244 | 43.7 | 0.035 | 0.612 | Leached |
| A7 | 3.25 | 8.13 | 0.000244 | 44.1 | 0.036 | 0.617 | Unleached |
| A10 | 3.43 | 8.31 | 0.000244 | 44.1 | 0.036 | 0.617 | Leached |
| A20 | 3.41 | 8.3 | 0.000244 | 44.2 | 0.036 | 0.618 | Unleached |
| A11 | 3.17 | 8.09 | 0.000244 | 44.4 | 0.036 | 0.622 | Leached |
| A8 | 3.04 | 7.98 | 0.000244 | 44.6 | 0.036 | 0.625 | Unleached |
| Average of | — | — | — | — | 0.035 | 0.612 | Leached |
| Leached | | | | | | | |
| Wood | | | | | | | |
| Substrates | | | | | | | |
| Average of | — | — | — | — | 0.036 | 0.614 | Unleached |
| Unleached | | | | | | | |
| Wood | | | | | | | |
| Substrates | | | | | | | |

The leaching results for wood substrates treated with the wood preservative comprising iron oxide and the wood substrates treated with the wood preservative comprising zinc sulfate are shown in Table 8.

TABLE 8
Leaching Results

| | Fe | Fe | Zn | Zn | |
|---|---|---|---|---|---|
| | (Leached) | (Unleached) | (Leached) | (Unleached) | % |
| Marker | (PPM) | (PPM) | (PPM) | (PPM) | Leached |
| Iron Oxide | 2277.5 | 2190.9 | 30.5 | 17.8 | −3.95% |
| (0.25%) | | | | | Iron |
| Zn Sulfate | 54.3 | 28.1 | 374.8 | 564.8 | 33.64% |
| (0.20%) | | | | | Zn |

The leaching results shown above in Table 8 indicate that iron in the iron oxide leaching was negligible since the amount of iron in the leached samples was higher than the amount of iron in the unleached samples. The amount of zinc that leached out of the zinc sulfate treated samples was 34%, which can be considered acceptable.

The examples illustrate that the method for determining a source of a wood product according to the present disclosure may be used to reliably determine the presence of iron and zinc in treated wood products. For example, Table 8 indicates that most of the iron and zinc in the treated wood products can be attributed to the markers in the respective wood preservative compositions used because the zinc levels in the iron oxide treated samples are substantially lower than the zinc levels in the zinc sulfate treated samples, and the iron levels in the zinc sulfate treated samples are substantially lower than the iron levels in the iron oxide treated samples. Thus, the above results indicate that the background levels of iron and zinc in wood substrates treated with CCA alone are low enough such that a specific metal content in a treated wood product quantitatively determined with an analytical measurement method can be used to reliably determine a presence of a marker compound within the treated wood product. Accordingly, the methods and compositions according to the present disclosure can provide the benefits of reliably determining the presence of a marker compound in a wood product with a visual assessment thereof shortly after time of manufacture and/or before any significant degradation, and with an analytical measurement when decay and/or degradation of the wood product due to long term field exposure would otherwise increase the level of uncertainty in results of a visual determination of the wood appearance and/or color.

In this specification, unless otherwise indicated, all numerical parameters are to be understood as being prefaced and modified in all instances by the term “about”, in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Also, any numerical range recited herein includes all sub-ranges subsumed within the recited range. For example, a range of “1 to 10” includes all sub-ranges between (and including) the recited minimum value of 1 and the recited maximum value of 10, that is, having a minimum value equal to or greater than 1 and a maximum value equal to or less than 10. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited.

The grammatical articles “a,” “an,” and “the,” as used herein, are intended to include “at least one” or “one or more,” unless otherwise indicated, even if “at least one” or “one or more” is expressly used in certain instances. Thus, the articles are used herein to refer to one or more than one (i.e., to “at least one”) of the grammatical objects of the article. Further, the use of a singular noun includes the plural, and the use of a plural noun includes the singular, unless the context of the usage requires otherwise.

Any patent, publication, or other disclosure material identified herein is incorporated by reference into this specification in its entirety unless otherwise indicated, but only to the extent that the incorporated material does not conflict with existing descriptions, definitions, statements, or other disclosure material expressly set forth in this specification. As such, and to the extent necessary, the express disclosure as set forth in this specification supersedes any conflicting material incorporated by reference. Any material, or portion thereof, that is said to be incorporated by reference into this specification, but which conflicts with existing definitions, statements, or other disclosure material set forth herein, is only incorporated to the extent that no conflict arises between that incorporated material and the existing disclosure material. Applicants reserve the right to amend this specification to expressly recite any subject matter, or portion thereof, incorporated by reference herein.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

One skilled in the art will recognize that the herein-described components, devices, operations/actions, and objects, and the discussion accompanying them, are used as examples for the sake of conceptual clarity and that various configuration modifications are contemplated. Consequently, as used herein, the specific examples/embodiments set forth and the accompanying discussion are intended to be representative of their more general classes. In general, use of any specific exemplar is intended to be representative of its class, and the non-inclusion of specific components, devices, operations/actions, and objects should not be taken limiting. While the present disclosure provides descriptions of various specific aspects for the purpose of illustrating various aspects of the present disclosure and/or its potential applications, it is understood that variations and modifications will occur to those skilled in the art. Accordingly, the invention or inventions described herein should be understood to be at least as broad as they are claimed and not as more narrowly defined by particular illustrative aspects provided herein.

What is claimed is:

1. A method for determining a source of a treated wood product, the method comprising:
detecting a presence of a marker in the treated wood product; and
determining the source of the treated wood product based on the detected presence of the marker.

2. The method of claim 1, wherein the marker comprises a metal oxide, a metal salt, or combinations thereof.

3. The method of claim 2, wherein the detecting the presence of the marker comprises determining a color of the treated wood product, measuring presence of the marker with an analytical instrument, contacting the treated wood product with a secondary indicator, or combinations thereof.

4. The method of claim 1, wherein the marker comprises zinc sulfate.

5. The method of claim 4, wherein the detecting the presence of the marker comprises contacting the treated wood product with a secondary indicator, wherein the treated wood product changes color in response to presence of the marker.

6. The method of claim 5, wherein the secondary indicator comprises a chelating agent.

7. The method of claim 5, wherein the secondary indicator comprises dithizone.

8. The method of claim 1, wherein the marker comprises iron oxide.

9. The method of claim 8, wherein the detecting the presence of the marker comprises determining a color of the treated wood product.

10. The method of claim 1, wherein the detecting the presence of the marker comprises measuring presence of the marker with an analytical instrument and wherein the analytical instrument utilizes X-ray fluorescence spectroscopy, atomic absorption spectroscopy, inductive coupled plasma spectroscopy, transmission electron microscopy, scanning electron microscopy, or combinations thereof.

11. The method of claim 1, further comprising, prior to detecting the presence of the marker, removing a portion of the treated wood product to expose a surface of the treated wood product.

12. The method of claim 11, wherein the removing the portion of the treated wood product comprises cutting, sawing, sanding, slicing, milling, machining, splitting, drilling, or combinations thereof.

13. A wood preservative composition comprising:
at least 0.1% by weight of chromated copper arsenate (CCA) based on a total weight of the wood preservative composition;
0.01% to 10% by weight of a metal salt and/or a metal oxide based on the total weight of the wood preservative composition; and
an aqueous solvent.

14. The wood preservative composition of claim 13, wherein a weight ratio of the CCA to the metal salt and/or metal oxide is in a range of 1:10 to 500:1.

15. The wood preservative composition of claim 13, wherein the wood preservative composition comprises 0.6% by weight to 6% by weight of the CCA based on the total weight of the wood preservative composition.

16. The wood preservative composition of claim 13, wherein the metal salt comprises zinc, iron, or combinations thereof.

17. The wood preservative composition of claim 13, wherein the metal salt comprises a metal sulfate, a metal chloride, a metal nitrate, or combinations thereof.

18. The wood preservative composition of claim 13, wherein the metal salt comprises zinc sulfate.

19. The wood preservative composition of claim 13, wherein the wood preservative composition comprises 0.01% to 3.0% by weight of the metal salt.

20. The wood preservative composition of claim 13, wherein the wood preservative composition further comprises a colorant, an ultraviolet stabilizer, a de-foamer, a water repellent, an additional biocide, a fungicide, a termiticide, a fire retardant, a mold inhibitor, a wax oil emulsion, or combinations thereof.

21. The wood preservative composition of claim 13, wherein the wood preservative composition has a pH in a range of 0.1 to 3.0 as measured at a temperature in a range of 20° C. to 30° C.

22. The wood preservative composition of claim 13, wherein the wood preservative composition is a concentrate.

23. The wood preservative composition of claim 13, wherein the wood preservative composition is a diluted composition.

24. A treated wood product produced by treating a wood substrate with the wood preservative composition of claim 13.

25. The treated wood product of claim 24, wherein the treated wood product retains at least 0.1 pounds of per cubic foot of total CCA active oxides as defined in 2023 AWPA Book of Standards.

26. The treated wood product of claim 24, wherein the treated wood product retains at least 10 grams of metal salt and/or metal oxide per cubic meter of the treated wood product.

27. The treated wood product of claim 24, wherein the metal salt and/or metal oxide exhibits a leachability of no greater than 40% from the wood product measured according to AWPA E11 leaching water study.

28. The treated wood product of claim 24, wherein the treated wood product comprises timber, plywood, laminated veneer lumber, cross laminated timber, parallel strand lumber, structural glued laminated timber, particle board, dimensional lumber, or combinations thereof.

29. The treated wood product of claim 24, wherein the treated wood product comprises a deck, a rail, a fence, a utility pole, a post, a pile, a railway tie, a railroad bridge, cladding, siding, or combinations thereof.

30. The treated wood product of claim 24, wherein the treated wood product comprises eucalyptus, southern pine, Douglas fir, Jack pine, red pine, Lodgepole pine, radiata pine, Alaska yellow cedar, Hem-fir, Nordic pine, Scotts pine, white spruce, Spruce-Pine-Fir, redwood, white oak, red oak, maple, black and red gum, Norway spruce, Sitka spruce, western red cedar, western larch, ponderosa pine, or combinations thereof.

31. A method for manufacturing a treated wood product, the method comprising
treating a wood substrate with the wood preservative composition of claim 13, thereby forming the treated wood product.

32. The method of claim 31, wherein the method comprises contacting the wood substrate with the wood preservative composition for a period of time suitable to inhibit fungal decay of the wood substrate.

33. The method of claim 32, wherein the contacting the wood substrate with the wood preservative composition comprises a pressure process, a vacuum process, or combinations thereof.
